(19) United States
(12) Patent Application Publication
(10) Pub. No.: US 2020/0211681 A1
(43) Pub. Date: Jul. 2, 2020

(54) METHOD AND A SYSTEM FOR ASSISTING ADVISER DURING USER EVALUATION

(71) Applicant: Wipro Limited, Bangalore (IN)

(72) Inventor: Manjunath Ramachandra Iyer, Bangalore (IN)

(21) Appl. No.: 16/281,966

(22) Filed: Feb. 21, 2019

(30) Foreign Application Priority Data
Dec. 29, 2018 (IN) 201841049823

(51) Int. Cl. G16H 10/60; G16H 15/00; G16H 50/30; A61B 5/00

(52) U.S. Cl. CPC G16H 10/60; G16H 15/00; G16H 20/00; A61B 5/0022; G16H 50/30

(57) ABSTRACT

A method and a system are described for assisting an adviser during user evaluation using a medical data processing system. The method includes classifying a medical record of a user into a plurality of data-segments based on a plurality of data layers. The method includes tagging the plurality of data-segments with associated data-segment parameters where the associated data-segment parameters indicates a context of each of the plurality of data segments. The method further includes determining one or more data-segments of the plurality of data-segments, in response to a conversation, wherein the one or more data-segments are determine based on a comparison of the context of each of the plurality of data segments with a context of the conversation. Further, the method includes rendering the determined one or more data-segments to the adviser based on the comparison.

### TECHNICAL FIELD

The present subject matter is related, in general, to healthcare informatics and more specifically, but not exclusively, to a method and a system for assisting adviser during user evaluation,

A medical evaluation and an efficient prognosis is a must precursor to any medical treatment. The field of medical science is laden with lot of medical jargons and medical data, and especially while treating a user, an adviser has to consider lots of data before diagnosing and hence prescribing a treatment for the user. While treating the user, the adviser has to consider past diseases, historical medical data and records and other data on chronic diseases, ailments and their treatments. In the wake of treating a current medical issue, the adviser may miss out on previous considerations, which if missed may interfere with a current evaluation and a treatment may in some extent be fatal to the user if there is a wrong prescription. Existing technology on electronic health records are handy, but they are handy only in recording and archiving conversations between the user and the adviser.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

According to embodiments illustrated herein, there may be provided a method of assisting an adviser during user evaluation using a medical data processing system. The method includes classifying a medical record of a user into a plurality of data-segments based on a plurality of data layers. The plurality of data layers includes a public data layer, a shareable-with-consent data layer and a private data layer. The method then includes tagging the plurality of data-segments with one or more associated data-segment parameters. The one or more associated data-segment parameters include at least one of one or more keywords, medical disorders, adviser, therapy prescribed, a start date and an end date associated with the therapy, and clinical tests undergone. The one or more associated data-segment parameters indicates a context of each of the plurality of data segments. The method then includes determining one or more data-segments of the plurality of data-segments, in response to a conversation. The one or more data-segments are determine based on a comparison of the context of each of the plurality of data segments with a context of the conversation. Further, the method includes rendering the determined one or more data-segments to the adviser based on the comparison.

According to embodiments illustrated herein, there may be provided a medical data processing system to assist the adviser during user evaluation. The medical data processing system includes a processor; and a memory communicatively coupled to the processor. The memory stores processor executable instructions, which on execution causes the processor to classify a medical record of a user into a plurality of data-segments based on a plurality of data layers. The plurality of data layers includes a public data layer, a shareable-with-consent data layer and a private data layer. The medical data processing system then tags the plurality of data-segments with one or more associated data-segment parameters. The one or more associated data-segment parameters include at least one of one or more keywords, medical disorders, adviser, therapy prescribed, a start date and an end date associated with the therapy, and clinical tests undergone. The one or more associated data-segment parameters indicates a context of each of the plurality of data segments. The medical data processing system then determines one or more data-segments of the plurality of data-segments, in response to a conversation. The one or more data-segments are determined based on a comparison of the context of each of the plurality of data segments with a context of the conversation. Further, the medical data processing system renders the determined one or more data-segments to the adviser based on the comparison.

According to embodiments illustrated herein, a non-transitory computer-readable storage medium having stored thereon, a set of computer-executable instructions for causing a computer comprising one or more processors to perform steps of classifying a medical record of a patient into a plurality of data-segments based on a plurality of data layers. The plurality of data layers includes a public data layer, a shareable-with-consent data layer and a private data layer. The computer-readable storage medium may tag the plurality of data-segments with one or more associated data-segment parameters. The one or more associated data-segment parameters include at least one of one or more keywords, medical disorders, medical practitioners, therapy prescribed, a start date and an end date associated with the therapy, and clinical tests undergone. The one or more associated data-segment parameters indicates a context of each of the plurality of data segments. The computer-readable storage medium may determine one or more data-segments of the plurality of data-segments, in response to a conversation. The one or more data-segments are determine based on a comparison of the context of each of the plurality of data segments with a context of the conversation. The computer-readable storage medium may render the determined one or more data-segments to a medical practitioner based on the comparison,

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates a block diagram of an exemplary environment, in which various embodiments of the present disclosure may function.

FIG. 2 illustrates a block diagram of a medical data processing system, in accordance with some embodiments of the present disclosure.

FIG. 3 is a flowchart illustrating a method of assisting adviser s during user evaluation.

FIG. 4 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

The present disclosure may be best understood with reference to the detailed figures and description set forth herein. Various embodiments are discussed below with reference to the figures. However, those skilled in the art readily appreciate that the detailed descriptions given herein with respect to the figures are simply for explanatory purposes as the methods and systems may well extend beyond the disclosed embodiments. For example, the teachings presented and the needs of a particular application may yield multiple alternative and suitable approaches to implement the functionality of any detail described herein.

References to “one embodiment,” “at least one embodiment,” “an embodiment” “one example,” “an example,” “for example,” and so on, indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” may not necessarily refer to the same embodiment.

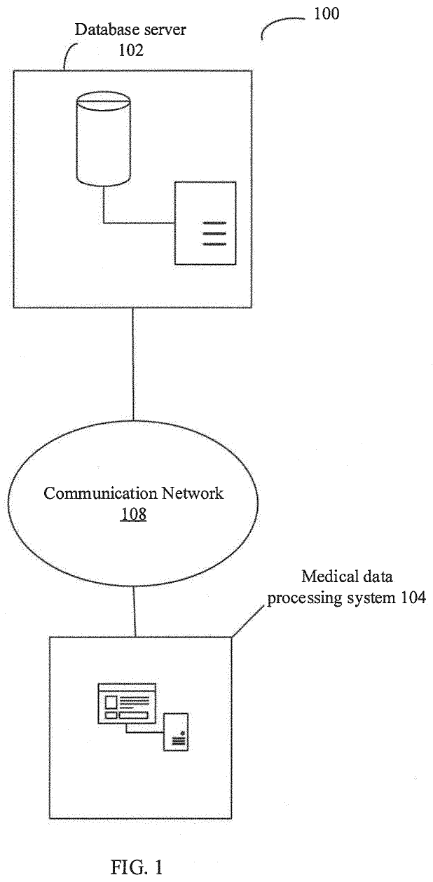

FIG. 1 is a block diagram that illustrates an exemplary environment 100 in which various embodiments of the present disclosure may function. The environment 100 may include a database server 102. In some embodiments, the database server 102 may be found embedded in the medical data processing system 104. The database server 102 may include applications that provides database services to the computing systems included in the medical data processing system 104. For example, the database server 102 may provide database management systems in form of database-server functionality, for example MySQL, while other database-server models, for example SQLite™, may provide functionalities of an embedded database. The database server 102 communicates via a communication network 108 to the medical data processing system 104.

The communication network 104 although represented as one communication network in FIG. 1 may in reality correspond to different communication networks under different contexts. For example, the communication network 104 may include various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), File Transfer Protocol (FTP), ZigBee™, EDGE™, infrared (IR), IEEE 802.11, 802.16, 2G, 3G, 4G cellular communication protocols, and/or Bluetooth™ (BT) communication protocols. The communication network 104 may include, but is not limited to, the Internet, a cloud network, a Wireless Fidelity (Wi-Fi) network, a Wireless Local Area Network (WLAN), a Local Area Network (LAN), a telephone line (POTS), and/or a Metropolitan Area Network (MAN).

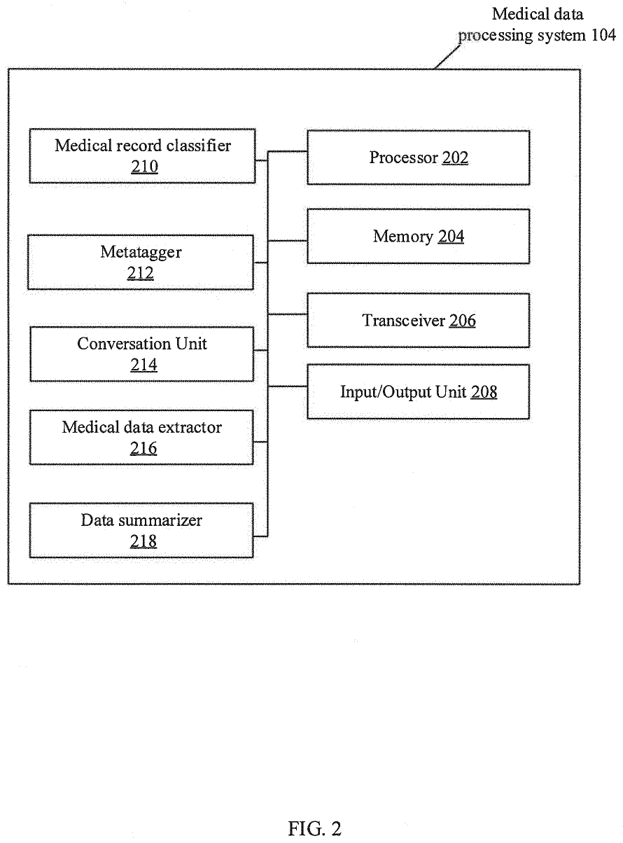

In an embodiment, the medical data processing system 104 may refer to a computing device that provides a method for layered organization of medical records and simultaneously maintain privacy of the medical record of the user by rendering one or more relevant segments from the medical record to the adviser. The medical data processing system 104 may include hardware and/or software that may be configured to perform one or more predetermined operations. And apart from the predetermined operations, the medical data processing system 104 has machine-learning capabilities. The medical data processing system 104 may refer to a computing device or a software framework hosting an application or a software service. The medical data processing system 104 may perform one or more operations through one or more units (explained in detail in FIG. 2).

In an embodiment, the medical data processing system 104 may execute procedures such as, but not limited to, programs, routines, or scripts stored in one or more memories for supporting the hosted application or the software service. In an embodiment, the hosted application or the software service may be configured to perform one or more predetermined operations. The medical data processing system 104 may be realized through various types of servers such as, but are not limited to, a Java application server, a .NET framework application server, a Base4 application server, a PHP framework application server, or any other application server framework,

FIG. 2 is a block diagram that illustrates the medical data processing system 104 configured for assisting the adviser during user evaluation. The medical data processing system 104 may include a processor 202, a memory 204, a transceiver 206, and an input/output unit 208. The medical data processing system 104 may further include a medical record classifier 210, a metatagger 212, a conversation unit 214, a medical data extractor 216 and a data summarizer unit 218. The processor 202 may be communicatively coupled to the memory 204, the transceiver 206, the input/output unit 208, the medical record classifier 210, the metatagger 212, the conversation unit 214, and the medical data extractor 216.

The processor 202 may include suitable logic, circuitry, interfaces, and/or code that may he configured to execute a set of instructions stored in the memory 204. The processor 202 may be implemented based on a number of processor technologies known in the art. Examples of the processor 202 include, but are not limited to, an X86-based processor, a Reduced Instruction Set Computing (RISC) processor, an Application-Specific Integrated Circuit (ASIC) processor, a Complex Instruction Set Computing (CISC) processor, and/or other processor.

The memory 204 may include suitable logic, circuitry, interfaces, and/or code that may be configured to store the set of instructions, which may be executed by the processor 202 for assisting the adviser during the user evaluation. In an embodiment, the memory 204 may be configured to store one or more programs, routines, or scripts that may be executed in coordination with the processor 202. The memory 204 may be implemented based on a Random Access Memory (RAM), a Read-Only Memory (ROM), a Hard Disk Drive (HDD), a storage server, and/or a Secure Digital (SD) card.

The transceiver 206 may receive the at least one medical record during the user evaluation. In an embodiment the medical record may be populated at least by capturing data from the user at a time of enrollment, or by linking the medical data processing system 104 to social security number of the user. Once the transceiver 206 receives the medical record, the medical record classifier 210 classifies the medical record of the user into a plurality of data-segments based on a plurality of data layers. Furthermore, the medical record classifier 210 may classify the plurality of data segments based on repetition of occurrence of the plurality of data segments in the medical record. The medical record classifier 210 includes machine learning capabilities to intelligibly classify the plurality of data-segments from the medical record into each of the respective data layers. This ability of classification is enabled by one or more sub-classifiers (not shown in the Figures). The sub-classifiers may be at least a generic classifier, a user centric classifier and medical issue-centric classifier. The sub-classifiers may respectively classify the plurality of data-segments into the plurality of data layers, namely, a public data layer, a shareable-with-consent data layer and a private data layer. For example, considering a scenario,

“Swarnil” had headache on Thursday . . . Swarnil had headache on Friday . . . Swarnil was down with headache on Saturday . . . . ”

The medical record classifier 210 will sub-classify “Swarnil” under the user-centric classifier as the user name, and sub-classify headache under the medical issue-centric classifier as the name of the chronic disease. In an embodiment, a neural network in the medical record classifier 210 may be configured to classify the medical issue as the chronic disease or occasionally encountered issue. For example, by checking the medical health record along with the dates of visit, it should be possible to tell if the disorder or medical issue is chronical or a first time case. Every time an issue is encountered, the bias is increased towards a node in the neural network, indicating the user has visited for 4 times with headache and he is likely to visit for the fifth time as well.

In another embodiment the aforementioned plurality of data layers include user data of increasing sensitivity, which can be accessed by the adviser or a non-adviser based on satisfying one or more data-segment parameters (later explained in detail). In the exemplary embodiment provided, the public data layer may include at least the user's name, nature of work, gender, and ethnicity. The shareable-with-consent data layer may include data on chronic disorders, insurance policies subscribed, social security number, salary range and residential or work address. The private data layer include data on family background, family related medical histories, acquired disorders, previous medical issues, prescriptions, medications taken, name of the adviser consulted and type of the adviser. Any person, for example the adviser or the non-adviser like a billing executive may have access to the plurality of data segments (aforementioned) from the medical record. In another embodiment, if an insurance company has to view the insurance policy related data-segment, the user's consent may allow the medical data processing system 104 to retrieve the data-segment from the shareable-with-consent data layer of the medical record. In another exemplary embodiment, the adviser may only view a data-segment associated with the private data layer while having a conversation with the user.

In an embodiment, the transceiver 206 may also he configured to receive comments from a user who is geographically distant from the adviser. For example, in an era of telemedicine the user who may be situated in a difficult terrain may conduct at least an audio conversation with the adviser. And not necessarily a face to face conversation with the adviser be always needed for the medical data processing system 104 to assist the adviser during the user evaluation.

The transceiver 206 may implement one or more known technologies to support wired or wireless communication with the communication network 104. In an embodiment, the transceiver 206 may include, but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. The transceiver 206 may communicate via wireless communication with networks, such as the Internet, an Intranet and/or a wireless network, such as a cellular telephone network, a wireless local area network (LAN) and/or a metropolitan area network (MAN). The wireless communication may use any of a plurality of communication standards, protocols and technologies, for example Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (Wi-Fi) (for example, IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

Once the medical record is classified into the plurality of data-segments based on the plurality of data layers, the metatagger 212 tags the plurality of data-segments with one or more associated data-segment parameters. The one or more associated data-segment parameters may include at least one of one or more keywords, medical disorders, adviser, type of adviser, therapy prescribed, a start date and an end date associated with the therapy, and clinical tests undergone The one or more associated data-segment parameters indicates a context of each of the plurality of data segments.

For example, Swarnil is a user who has headache as a chronic medical issue. Earlier, he had made regular visits to his adviser Dr. Monali, a general physician, who had prescribed analgesic pills like paracetamol. However, Swarnil's comments (also in the medical record) suggest that the medications prescribed were ineffective. In spite of the prescriptions he has complained of headache as an ensuing issue. He was then prescribed an MRI scan to ascertain any brain related medical issue. In the exemplary embodiment, the metatagger 212 will tag “headache” as a medical disorder, and a chronic disorder, “Swarnil” as the user, “Dr. Monali” as the adviser, and “general physician” as the type of adviser, “paracetamol” as the prescribed medication and the prescribed “MRI scan” tagged as clinical tests undergone. And as mentioned, in a different embodiment the other each of the plurality of data-segments in their respective data layers will be tagged to respective data-segment parameters. In a further embodiment, the one or more data-segment parameters indicates the context of each of the plurality of data segments.

In the aforementioned embodiment, the plurality of data-segment parameters like “MRI scan”, “general physician”, “paracetamol” are gagged to respective data-segment parameters and are then respectively classified into sub-layers of the data layers, which is the private data layer in this exemplary case. And if an insurance company executive talks to the user regarding insurance policy number, which is tagged to a different layer, a data-segment tagged to insurance policies will only be extracted.

Once the plurality of data-segments is tagged with the one or more associated data-segment parameters, the conversation unit 214 determines the one or more data-segments of the plurality of data-segments, in response to a conversation, where the one or more data-segments are determined based on a comparison of the context of each of the plurality of data segments with a context of the conversation. The conversation may be a face to face conversation, or a tele-conversation or a natural language processing conversation with the medical data processing system 104.

For example, considering a conversation between the user and the insurance company executive,
“Swarnil—My name is Swarnil and I have completed treatment.
Insurance executive—Can you please provide more details on your treatment or medical issue?
Swarnil—My treatment was for skin rashes. The adviser had prescribed a few lotions and a few capsules for internal consumption. The total amount billed was Rupees 2000.
(It is to be noted that the user did not mention the insurance policy number and related details.)
Insurance executive—Can I retrieve the policy related documents?
Swarnil—Yes
(The medical data processing system 104 is configured with voice recognition capabilities for the user.)
Insurance Executive—So, here I can see the insurance policy related documents. But what was your recent medical issue about?

In the aforementioned exemplary conversation, the conversation unit 214 can parse the entire conversation constituently into one or more words. The parsed words like “Swarnil”, “treatment”, “insurance policy”, “insurance policy executive”, indicates the context of the conversation.

Once the plurality of data-segments is tagged with the one or more associated data-segment parameters, the medical data extractor 216 determines and then extracts the one or more data-segments of the plurality of data-segments. The one or more data-segments are determined based on a comparison of the context of each of the plurality of data segments with a context of the conversation. Determination of the context of the context of the conversation is based on parsing the conversation into at least one or more words or phrases and then comparing the at least one or more words or phrases with the one or more associated data-segment parameters. The conversation may be between the user and the adviser or between the user and the non-adviser like a billing executive, or an insurance company executive. In the aforementioned conversation, the parsed words, for example “treatment”, “skin rashes”, are tagged to associated data-segment parameter medical issue. Likely, “Swami!” is identified as the user, “lotion” is identified to the prescription.

Once the one or more data-segments of the plurality of data-segments is determined, the data summarizer unit 218 renders the determined one or more data-segments to the adviser based on the comparison. The one or more data-segments determined may be chunks of data in words or phrases and may often be difficult for the adviser to understand such data segments rendered. The summarizer unit 218 negates this ambiguity by rendering a complete grammatical sentence, including the determined one or more data-segments in the complete grammatical sentence. The summarizer unit 218 has an inbuilt grammar correction engine (not shown in the figures) for augmenting in sentence construction. The inbuilt grammar correction engine may be pre-trained for sentence construction. For example, during a conversation the adviser may talk to the user regarding any past surgery. It may so happen that the user had a surgery one-month prior from the date of the current conversation. The summarizer unit 218 may render a sentence, which may read “The user had a surgery dated 26th of Nov. 2018.”

In another embodiment, the adviser asked the user if he has abnormal blood pressure, to which the user answered just in the affirmative. However, the medical data processing system 104 found that the user has been suffering from high blood pressure for the past 5 years along with the medications prescribed to him by advisers consulted earlier. This information rendered, shall definitely help the current adviser in prescribing future medications. In yet another embodiment, the adviser prescribed the user antibiotic tablets, but was not aware of the fact that the user was severely allergic to antibiotic medications. The medical data processing system 104 after listening to the prescription could render an information from the medical record that the user is allergic to antibiotics. This action alerts the adviser to alter the medication.

In an alternative embodiment, the adviser may converse with the user who is differently-abled. For example, the adviser may converse with a user with highly subdued vocal ability. The user may converse via sign language and in this case along with the voice recognition engine, the medical data processing system 104 may also be configured to receive affirmative or negative inputs from the user on a touch display, in turn configured to recognize unique biometric inputs of the respective user. The medical data processing system 104 may continue to render the one or more determined data-segments, as the adviser converses with the differently-abled user and as the differently-abed user continues to provide one or more inputs via media comfortable to him/her. In an embodiment the medical data processing system 104 can be further configured to update the medical record based on the adviser's evaluation of the user. For example, the adviser may find that an earlier set of medicines prescribed to the user are not henceforth suitable and as a result the user needs to be subjected to new set of medicines. After the prescribing, the adviser may update the medical record. The medical record shall then include the earlier set of medicines prescribed and also the new set of medicines currently prescribed.

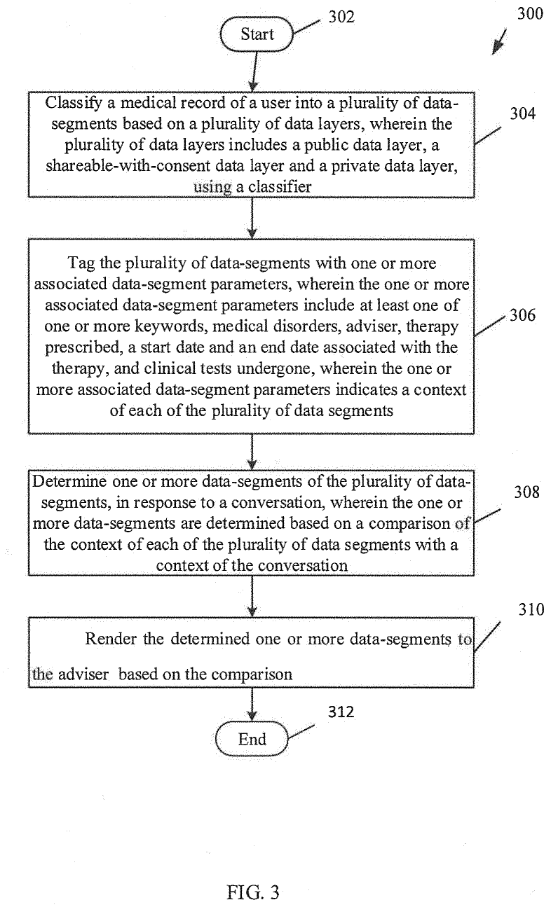

FIG. 3 illustrates a flow diagram of a method 300 of assisting an adviser during user evaluation. The method 300 starts at step 302. At step 304 the medical data processing system 104 may be configured to classify a medical record of a user into a plurality of data-segments based on a plurality of data layers, wherein the plurality of data layers includes a public data layer, a shareable-with-consent data layer and a private data layer. At step 306 the medical data processing system 104 may be configured to tag the plurality of data-segments with one or more associated data-segment parameters. The one or more associated data-segment parameters include at least one of one or more keywords, medical disorders, adviser, therapy prescribed, a start date and an end date associated with the therapy, and clinical tests undergone, wherein the one or more associated data-segment parameters indicates a context of each of the plurality of data segments. At step 308 the medical data processing system 104 may be configured to determine one or more data-segments of the plurality of data-segments, in response to a conversation, wherein the one or more data-segments are determined based on a comparison of the context of each of the plurality of data segments with a context of the conversation. At step 310 the medical data processing system 104 may be configured to render the determined one or more data-segments to the adviser based on the comparison. The method 300 ends at step 312.

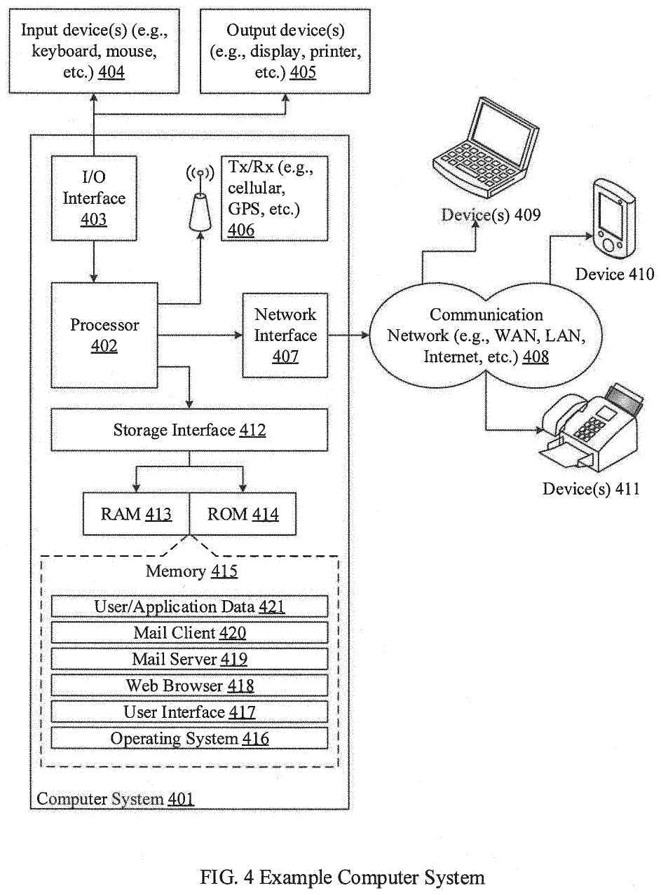

FIG. 4 illustrates a block diagram of an exemplary computer system for implementing embodiments consistent with the present disclosure. Variations of computer system 401 may be used for assisting the adviser during user evaluation using the medical data processing system 104. The computer system 401 may include a central processing unit (“CPU” or “processor”) 402. Processor 402 may include at least One data processor for executing program components for executing user or system-generated requests. A user may include a person, a person using a device such as such as those included in this disclosure, or such a device itself. The processor may include specialized processing units such as integrated system (bus) controllers, memory management control units, floating point units, graphics processing units, digital signal processing units, etc. The processor 402 may include a microprocessor, such as AMD Athlon™, Duron or Opteron, ARM'S application, embedded or secure processors, IBM PowerPC®, Intel's Core, Itaniurn, Xeon, Celeron or other line of processors, etc. The processor 402 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 402 may be disposed in communication with one or more input/output (I/O) devices via I/O interface 403. The I/O interface 403 may employ communication protocols/methods such as, without limitation, audio, analog, digital, monoaural, RCA, stereo, IEEE-I394, serial bus, universal serial bus (USB), infrared, PS/2, BNC, coaxial, component, composite, digital visual interface (DVI), high-definition multimedia interface (IMMO, RF antennas, S-Video, VGA, IEEE 802.n /b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface 403, the computer system 401 may communicate with one or more I/O devices. For example, the input device 404 may be an antenna, keyboard, mouse, joystick, (infrared) remote control, camera, card reader, fax machine, dongle, biometric reader, microphone, touch screen, touchpad, trackball., sensor (e.g., accelerometer, light sensor, GPS, gyroscope, proximity sensor, or the like), stylus, scanner, storage device, transceiver, video device/source, visors, etc. Output device 405 may be a printer, fax machine, video display (e.g., cathode ray tube (CRT), liquid crystal display (LCD), light-emitting diode (LED), plasma, or the like), audio speaker, etc. In some embodiments, a transceiver 406 may be disposed in connection with the processor 402. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G FISDPA/FISUPA communications, etc.

In some embodiments, the processor 402 may be disposed in communication with a communication network 408 via a network interface 407. The network interface 407 may communicate with the communication network 408. The network interface may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network 408 may include, without limitation, a direct interconnection, local area network (LAN), wide area network (WAN), wireless network (e.g., using Wireless Application Protocol), the Internet, etc. Using the network interface 407 and the communication network 408, the computer system 401 may communicate with devices 410, 411, and 412. These devices may include, without limitation, personal computer(s), server(s), fax machines, printers, scanners, various mobile devices such as cellular telephones, smartphones (e.g., Apple iPhone™, Smart TV, Android-based phones, etc.), tablet computers, eBook readers (Amazon Kindle, Nook, etc.), laptop computers, notebooks, gaming consoles (Microsoft Xbox™, Nintendo DS™, Sony PlayStation™, etc.), or the like. In some embodiments, the computer system 701 may itself embody one or more of these devices.

In some embodiments, the processor 402 may be disposed in communication with one or more memory devices (e.g., RAM 413, ROM 414, etc.) via a storage interface 412. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

The memory devices may store a collection of program or database components, including, without limitation, an operating system 416, user interface 417, web browser 418, mail server 419, mail client 420, user/application data 421 (e.g., any data variables or data records discussed in this disclosure), etc. The operating system 416 may facilitate resource management and operation of the computer system 401. Examples of operating systems include, without limitation, Apple Macintosh OS X, UNIX, Unix-like system distributions (e.g., Berkeley Software Distribution™ (BSD), FreeBSD, NetBSD, OpenBSD, etc.), Linux distributions (e.g., Red Hat™, Ubuntu™, Kubuntu™, etc.), IBM OS/2™, Microsoft Windows™ (XP, Vista/7/8, etc.), Apple iOS®, Google Android™, or the like. User interface 417 may facilitate display, execution, interaction, manipulation, or operation of program components through textual or graphical facilities. For example, user interfaces may provide computer interaction interface elements on a display system operatively connected to the computer system 401, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows Aero™, Metro™, etc.), Unix X-Windows, web interface libraries (e.g., ActiveX™, Java™, Javascrip™, AJAX, HTML, Adobe Flash™, etc.), or the like.

In some embodiments, the computer system 401 may implement a web browser 418 stored program component. The web browser may be a hypertext viewing application, such as Microsoft™ Internet Explorer™, Google Chrome™, Mozilla Firefox™, Apple Safari™, etc. Secure web browsing may be provided using HTTPS (secure hypertext transport protocol), secure sockets layer (SSL), Transport Layer Security (TLS), etc. Web browsers may utilize facilities such as AJAX, DHTML, Adobe Flash, JavaScript, Java, application programming interfaces (APIs), etc. In some embodiments, the computer system 401 may implement a mail server 419 stored program component. The mail server may be an Internet mail server such as Microsoft Exchange™, or the like. The mail server may utilize facilities such as ASP, ActiveX, ANSI C++/C#, Microsoft .NET™, CGI scripts, Java™, JavaScript™, PFRL™, PHP™, Python™, WebObjects, etc. The mail server may utilize communication protocols such as internet message access protocol (IMAP), messaging application programming interface (MAPI), Microsoft Exchange, post office protocol (POP), simple mail transfer protocol (SMTP), or the like. In some embodiments, the computer system 401 may implement a mail client 420 stored program component. The mail client may be a mail viewing application, such as Apple Mail™, Microsoft Entourage™, Microsoft Outlook™, Mozilla Thunderbird™, etc.

In some embodiments, computer system 401 may store user/application data 421, such as the data, variables, records, etc. as described in this disclosure. Such databases may be implemented as fault-tolerant, relational, scalable, secure databases such as Oracle™ or Sybas™. Alternatively, such databases may be implemented using standardized data structures, such as an array, hash, linked list, struct, structured text file (e.g., XML), table, or as object-oriented databases. Such databases may be consolidated or distributed, sometimes among the various computer systems discussed above in this disclosure. It is to be understood that the structure and operation of the any computer or database component may be combined, consolidated, or distributed in any working combination.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media,

The advantages of the present disclosure address the requirement of assisting an adviser during user evaluation using the medical data processing system. A adviser while prescribing remedies for an ailing user must keep a lot of user related medical considerations, which if prescribed might affect the health of the user. The medical data processing system claimed thus assists the adviser during evaluation in wake that the adviser does not miss-out on important data before prescribing medications for the user.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise. The terms “including”, “comprising” “having” and variations thereof mean “including but not limited to”, unless expressly specified otherwise. The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on, Accordingly, the embodiments of the present invention are intended to be illustrative, but not limiting, of the scope of the invention, which is set forth in the following claims. While various aspects and embodiments have been disclosed herein, other aspects and embodiments will he apparent to those skilled in the art. The various aspects and embodiments disclosed herein are for purposes of illustration and are not intended to be limiting, with the true scope and spirit being indicated by the following claims.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that includes a portion of an integrated circuit that also performs other functions.

A person with ordinary skills in the art will appreciate that the systems, modules, and sub modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

We claim:

1. A method of assisting an adviser during user evaluation, the method comprising:
classifying, by a medical data processing system, a medical record of a user into a plurality of data-segments based on a plurality of data layers, wherein the plurality of data layers includes a public data layer, a shareable-with-consent data layer and a private data layer;
tagging, by the medical data processing system, the plurality of data-segments with one or more associated data-segment parameters, wherein the one or more associated data-segment parameters include at least one of one or more keywords, medical disorders, adviser s, therapy prescribed, a start date arid an end date associated with the therapy, and clinical tests undergone, wherein the one or more associated data-segment parameters indicates a context of each of the plurality of data segments;
determining, by the medical data processing system, one or more data-segments of the plurality of data-segments, in response to a conversation, wherein the one or more data-segments are determined based on a comparison of the context of each of the plurality of data segments with a context of the conversation.
rendering, by the medical data processing system, the determined one or more data-segments to the adviser based on the comparison.

2. The method of claim I, further comprising a classifier to classify the plurality of data segments into each of the public data layer, the shareable-with-consent data layer and the private data layer.

3. The method of claim 2, further comprising classifying the plurality of data segments is based on repetition of occurrence of the plurality of data segments in the medical record.

4. The method of claim 1, wherein the conversation generated is based on at least a face to face conversation, or a tele-conversation or a natural language processing conversation with the medical data processing system.

5. The method of claim 1, wherein determination of the one or more data-segments is based on:
parsing the conversation into at least one or more words or phrases; and
comparing the at least one or more words or phrases with the one or more associated data-segment parameters.

6. The method of claim 1, further comprising updating the medical record based on the adviser's evaluation of the user.

7. A medical data processing system for assisting an adviser during user evaluation, the medical data processing system comprising:
a processor; and
a memory communicatively coupled to the processor, wherein the memory stores processor executable instructions, which on execution causes the processor to:
classify a medical record of a user into a plurality of data-segments based on a plurality of data layers, wherein the plurality of data layers includes a public data layer, a shareable-with-consent data layer and a private data layer, using a classifier;
tag the plurality of data-segments with one or more associated data-segment parameters, wherein the one or more associated data-segment parameters include at least one of one or more keywords, medical disorders, adviser, therapy prescribed, a start date and an end date associated with the therapy, and clinical tests undergone, wherein the one or more associated data-segment parameters indicates a context of each of the plurality of data segments;
determine one or more data-segments of the plurality of data-segments, in response to a conversation, wherein the one or more data-segments are determined based on a comparison of the context of each of the plurality of data segments with a context of the conversation.
render the determined one or more data-segments to the adviser based on the comparison.

8. The medical data processing system of claim 7, further comprising classifying the plurality of data segments is based on repetition of occurrence of the plurality of data segments in the medical record and wherein determination of the one or more data-segments is based on:
parsing the conversation into at least one or more words or phrases; and
comparing the at least one or more words or phrases with the one or more associated data-segment parameters.

9. The medical data processing system of claim 7, wherein the conversation generated is based on at least a face to face conversation, or a tele-conversation or a natural language processing conversation with the medical data processing system.

10. The medical data processing system of claim 7, further comprising updating the medical record based on the adviser's evaluation of the user.

11. A non-transitory computer-readable medium storing computer-executable instructions for:
classifying a medical record of a user into a plurality of data-segments based on a plurality of data layers, wherein the plurality of data layers includes a public data layer, a shareable-with-consent data layer and a private data layer;
tagging the plurality of data-segments with one or more associated data-segment parameters, wherein the one or more associated data-segment parameters include at least one of one or more keywords, medical disorders, adviser's, therapy prescribed, a start date and an end date associated with the therapy, and clinical tests undergone, wherein the one or more associated data-segment parameters indicates a context of each of the plurality of data segments;
determining one or more data-segments of the plurality of data-segments, in response to a conversation, wherein the one or more data-segments are determined based on a comparison of the context of each of the plurality of data segments with a context of the conversation;
rendering the determined one or more data-segments to the adviser based on the comparison.
